(19) United States
(12) Patent Application Publication
(10) Pub. No.: US 2013/0236573 A1
(43) Pub. Date: Sep. 12, 2013

(54) ESKETAMINE FOR THE TREATMENT OF TREATMENT-REFRACTORY OR TREATMENT-RESISTANT DEPRESSION

(71) Applicant: JANSSEN PHARMACEUTICA NV, Beerse (BE)

(72) Inventors: Jaskaran Singh, Flemington, NJ (US); Ivo Caers, Beerse (BE)

(73) Assignee: JANSSEN PHARMACEUTICA NV, Beerse (BE)

(21) Appl. No.: 13/795,454

(22) Filed: Mar. 12, 2013

Related U.S. Application Data
(60) Provisional application No. 61/609,641, filed on Mar. 12, 2012.
(60) Provisional application No. 61/610,058, filed on Mar. 13, 2012.

(51) Int. Cl. A61K 31/135; A61K 45/06

(52) U.S. Cl. CPC A61K 31/135; A61K 45/06
USPC 424/730; 514/647; 514/217; 514/450; 514/211.13; 514/253.04; 514/438; 514/469; 514/321; 424/734; 514/253.07; 514/220; 514/259.41; 564/307

(57) ABSTRACT

The present invention is directed to methods for the treatment of treatment-refractory depression or treatment-resistant depression comprising administering to a patient in need thereof, a therapeutically effective amount of esketamine as mono-therapy or as combination therapy with at least on antidepressant.

### CROSS REFERENCE TO RELATED APPLICATIONS

The present application claims benefit of U.S. Provisional Patent Application Ser. No. 61/609,641, filed on Mar. 12, 2012, and U.S. Provisional Patent Application Ser. No. 61/610,058, filed on Mar. 13, 2012, which are incorporated herein by reference in their entirety and for all purposes.

### FIELD OF THE INVENTION

The present invention is directed to methods for the treatment of treatment-refractory depression or treatment-resistant depression comprising administering to a patient in need thereof, a therapeutically effective amount of esketamine as mono-therapy or as combination therapy with at least one antidepressant.

### BACKGROUND OF THE INVENTION

Major Depressive Disorder is defined as the presence of one of more major depressive episodes that are not better accounted for psychotic disorder or bipolar disorder. A major depressive episode is characterized by meeting five or more of the following criteria during the same 2 week period which represent a change in functioning and include at least depressed/sad mood or loss of interest and pleasure, indifference or apathy, or irritability and is usually associated with a change in a number of neurovegetative functions, including sleep patterns, appetite and body weight, motor agitation or retardation, fatigue, impairment in concentration and decision making, feelings of shame or guilt, and thoughts of death or dying (Diagnostic and Statistical Manual of Mental Disorders, 4^{th} Edition-TR, American Psychiatric Association, 2004; Harrison's Principles of Internal Medicine, 2000). Symptoms of a depressive episode include depressed mood; markedly diminished interest or pleasure in all, or almost all, activities most of the day; weight loss when not dieting or weight gain, or decrease or increase in appetite nearly every day; insomnia or hypersomnia nearly every day; psychomotor agitation or retardation nearly every day; fatigue or loss of energy nearly every day; feelings of worthlessness or excessive or inappropriate guilt nearly every day; diminished ability to think or concentrate, or indecisiveness, nearly every day; recurrent thoughts of death, recurrent suicidal ideation without a specific plan, or a suicide attempt or a specific plan for committing suicide. Further, the symptoms cause clinically significant distress or impairment in social, occupational, or other important areas of functioning. (Diagnostic and Statistical Manual of Mental Disorders, 4^{th} Edition-TR, American Psychiatric Association, 2004)

Current treatment options for unipolar depression include monotherapy or combination therapy with various classes of drugs including mono-amine oxidase inhibitors (MAOI), tricyclic antidepressants (TCA), serotonin specific reuptake inhibitors (SSRI), serotonin noradrenergic reuptake inhibitors (SNRI), noradrenaline reuptake inhibitor (NRI), “natural products” (such as Kava-Kava, St. John's Wort), dietary supplement (such as s-adenosylmethionine) and others. More specifically, drugs used in the treatment of depression include, but are not limited to imipramine, amitriptyline, desipramine, nortriptyline, doxepin, protriptyline, trimipramine, maprotiline, amoxapine, trazodone, bupropion, chlomipramine, fluoxetine, citalopram, escitalopram, sertraline, paroxetine, tianeptine, nefazadone, venlafaxine, desvenlafaxine, duloxetine, reboxetine, mirtazapine, phenelzine, tranylcypromine, and/or moclobemide. Several of these agents including, but not limited to, serotonin reuptake inhibitors are also used when depression and anxiety co-exist, such as in anxious depression.

In the clinic, 40-50% of depressed patients who are initially prescribed antidepressant therapy do not experience a timely remission of depression symptoms. This group typifies level 1 treatment-resistant depression, that is, a failure to demonstrate an “adequate” response to an “adequate” treatment trial (that is, sufficient intensity of treatment for sufficient duration). Moreover, about approximately 30% of depressed patients remain partially or totally treatment-resistant to at least two antidepressant treatments including combination treatments. Increasingly, treatment of treatment-resistant depression includes augmentation strategies including treatment with pharmacological agents such as, antipsychotics (such as quetiapine, aripiprazole, olanzapine, risperidone, and the like), lithium, carbamazepine, and triiodothyronine, and the like; adjunctive electroconvulsive therapy; adjunctive transcranial magnetic stimulation; deep brain stimulation, etc.

Ketamine (a racemic mixture of the corresponding S- and R-enantiomers) is an NMDA receptor antagonist, with a wide range of effects in humans, including analgesia, anesthesia, hallucinations, dissociative effects, elevated blood pressure and bronchodilation. Ketamine is primarily used for the induction and maintenance of general anesthesia. Other uses include sedation in intensive care, analgesia (particularly in emergency medicine) and treatment of bronchospasms. Ketamine has also been shown to be efficacious in the treatment of depression (particularly in those who have not responded to other anti-depressant treatment). In patients with major depressive disorders, ketamine has additionally been shown to produce a rapid antidepressant effect, acting within two hours.

The S-ketamine enantiomer (or S-(+)-ketamine or esketamine) has higher potency or affinity for the NMDA reception and thus potentially allowing for lower dosages; and is available for medical use under the brand name KETANEST S.

PAUL, R., et al., “Comparison of racemic ketamine and S-ketamine in treatment-resistant major depression: report of two cases”, World J. of Bio. Psych., 2009, pp 241-244, Vol. 10(3) describe two cases studies in which patients with a history of recurrent major depression were treated with intravenous of ketamine and S-ketamine.

PASKALIS, G., et al., “Oral Administration of the NMDA Receptor Antagonist S-Ketamine as Add-on Therapy of Depression: A Case Series”, Pharmacopsychiatry, 2010, pp 33-35, Vol. 40 present four case studies where depressed patients received 1.25 mg/kg oral S-ketamine as add-on to standard antidepressant therapy.

NOPPERS, I., et al., “Absence of long-term analgesic effect from a short-term S-ketamine infusion on fibromyalgia pain: A randomized, prospective, double blind, active placebo-controlled trial”, Eur. J. of Pain., 2011, article in press, describe a trial assessing the analgesic efficacy of S-(+)-ketamine on fibromyalgia pain.

MATTHEWS, S. J., et al., “Ketamine for Treatment-Resistant Unipolar Depression”, CNS Drugs, 2012, pp 1-16, provide a review of emerging literature on ketamine and a review of the pharmacology of both ketamine and S-ketamine.

There remains a need to provide an effective treatment for depression, particularly in patients with treatment-refractory or treatment-resistant depression.

### SUMMARY OF THE INVENTION

The present invention is directed to methods for the treatment of treatment-refractory or treatment-resistant depression, comprising administering to a patient in need thereof, a therapeutically effective amount of esketamine.

The present invention is further directed to a method for the treatment of treatment-refractory or treatment-resistant depression (TRD) comprising administering to a patient in need thereof, combination therapy with a therapeutically effective amount of esketamine and at least one antidepressant, as herein defined.

In an embodiment, the antidepressant(s) are each independently selected from the group consisting of mono-amine oxidase inhibitors, tricyclic antidepressants, serotonin specific reuptake inhibitors, serotonin noradrenergic reuptake inhibitors, noradrenaline reuptake inhibitors, natural products, dietary supplements, neuropeptides, compounds targeting neuropeptide receptors and hormones.

In an embodiment of the present invention is a method for the treatment of treatment-refractory or treatment-resistant depression (TRD) comprising administering to a patient in need thereof a therapeutically effective amount of esketamine in combination with one or more compounds selected from the group consisting of mono-amine oxidase inhibitors (MAOI) such as irreversible MAOI (phenelzine, tranylcypromine), reversible (MOAI) moclobemide, and the like; tricyclics such as imipramine, amitriptyline, desipramine, nortriptyline, doxepin, protriptyline, trimipramine, chlomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, fluvoxamine, and the like; serotonin receptor antagonists such as nefazadone, tianeptine and the like; serotonin noradrenergic reuptake inhibitors such as venlafaxine, milnacipran and the like; noradrenergic and specific serotonergic agents such as mirtazapine, and the like; noradrenaline reuptake inhibitors such as reboxetine, and the like; atypical antidepressants such as bupropion and the like, and the like; lithium, triple reuptake inhibitors, natural products such as Kava-Kava, St. John's Wort, and the like; dietary supplements such as s-adenosylmethionine and scopolamine, and the like; and neuropeptides such as thyrotropin-releasing hormone and the like, and the like; compounds targeting neuropeptide receptors such as neurokinin receptor antagonists and the like; and hormones such as triiodothyronine, and the like.

In an embodiment of the present invention is a method for the treatment of treatment-refractory or treatment-resistant depression (TRD) comprising administering to a patient in need thereof a therapeutically effective amount of esketamine in combination with one or more compounds selected from the group consisting of mono-amine oxidase inhibitors; tricyclics; tetracyclics; non-cyclics; triazolopyridines; serotonin reuptake inhibitors; serotonin receptor antagonists; serotonin noradrenergic reuptake inhibitors; serotonin noradrenergic reuptake inhibitors; noradrenergic and specific serotonergic agents; noradrenaline reuptake inhibitors; atypical antidepressants; natural products; dietary supplements; neuropeptides; compounds targeting neuropeptide receptors; and hormones.

Preferably, esketamine is administered in combination with one or more compounds selected from the group consisting of mono-amine oxidase inhibitors, tricyclics, serotonin reuptake inhibitors, serotonin noradrenergic reuptake inhibitors; noradrenergic and specific serotonergic agents and atypical antidepressants. More preferably, esketamine is administered in combination with one or more compounds selected from the group consisting of mono-amino oxidase inhibitors, tricyclics and serotonin reuptake inhibitors. More preferably, esketamine is administered in combination with one or more compounds selected from the group consisting of serotonin reuptake inhibitors.

In an embodiment, the present invention is a directed to a method for the treatment of treatment-refractory or treatment-resistant depression comprising administering to a patient in need thereof a therapeutically effective amount of esketamine in combination with one or more compounds selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, sertraline, paroxetine, citalopram, fluvoxamine, venlafaxine, desvenlafaxine, duloxetine, milnacipran, mirtazapine, bupropion, lithium, thyrotropin-releasing hormone and triiodothyronine.

Preferably, esketamine is administered in combination with one or more compounds selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, sertraline, paroxetine, citalopram, fluvoxamine, venlafaxine, milnacipran, mirtazapine and bupropion. More preferably, esketamine is administered in combination with one or more compounds selected from the group consisting of phenelzine, tranylcypromine, moclobemide, imipramine, amitriptyline, desipramine, nortiptyline, doxepin, protriptyline, trimipramine, chlomipramine, amoxapine, fluoxetine, sertraline, paroxetine, citalopram, escitalopram and fluvoxamine. More preferably, esketamine is administered in combination with one or more compounds selected from the group consisting of fluoxetine, sertraline, paroxetine, citalopram, escitalopram and fluvoxamine.

In an embodiment, the present invention is directed to a method for the treatment of treatment-refractory or treatment-resistant depression comprising administering to a patient in need thereof a therapeutically effective amount of esketamine in combination with one or more compounds selected from the group consisting of neuropeptides such as thyrotropin-releasing hormone and the like; compounds targeting neuropeptide receptors such as neurokinin receptors antagonists and the like; and hormones such as triiodothyronine and the like.

In an embodiment, the present invention is directed to a method for the treatment of treatment-refractory or treatment-resistant depression (TRD) comprising administering to a patient in need thereof, combination therapy with a therapeutically effective amount of esketamine, at least one antidepressant, and at least one atypical antipsychotic, as herein defined.

In an embodiment, the present invention is directed to a method for the treatment of treatment-refractory or treatment-resistant depression (TRD) comprising administering to a patient in need thereof, combination therapy with a therapeutically effective amount of esketamine, at least one antidepressant, and at least one atypical antipsychotic selected from the group consisting of quetiapine, aripiprazole, olnazapine, risperidone and paliperidone.

The present invention is further directed to the use of esketamine in the preparation of a medicament for treating treatment-refractory or treatment-resistant depression, in a patient in need thereof.

The present invention is further directed to esketamine for use in a method for the treatment of treatment-refractory or treatment-resistant depression, in a subject in need thereof.

In another embodiment, the present invention is directed to a composition comprising esketamine for the treatment of treatment-refractory or treatment-resistant depression.

### DETAILED DESCRIPTION OF THE INVENTION

The present invention is directed to methods for the treatment of treatment-refractory or treatment-resistant depression, comprising administering to a patient in need thereof, a therapeutically effective amount of esketamine.

The present invention is further directed to methods for the treatment of treatment-refractory or treatment-resistant depression comprising administering to a patient in need thereof, combination therapy comprising esketamine and at least one antidepressant.

In certain embodiments of the present invention, esketamine may be administered in combination with one or more antidepressants, as herein described, preferably in combination with one to three antidepressants, more preferably in combination with one to two antidepressants.

In certain embodiments of the present invention, esketamine may be administered in combination with one or more antidepressants, and further in combination with one or more atypical antipsychotics, herein described.

In an embodiment, the present invention is directed to combination therapy comprising esketamine and one or more antidepressants; wherein the esketamine is administered as acute treatment. In another embodiment, the present invention is directed to combination therapy comprising esketamine and one or more antidepressants wherein the esketamine is administered as acute treatment and wherein the one or more antidepressants are administered as chronic treatment. In another embodiment, the present invention is directed to combination therapy comprising esketamine and one or more antidepressants wherein the esketamine is administered as treatment to a patient in need thereof. In another embodiment, the present invention is directed to combination therapy comprising esketamine and one or more antidepressants wherein the esketamine is administered to a patient in need thereof during continuation treatment. In another embodiment, the present invention is directed to combination therapy comprising esketamine and one or more antidepressants wherein the esketamine is administered to a patient in need thereof during maintenance treatment.

As used herein, unless otherwise noted, the term “esketamine” shall mean the (S)-enantiomer of ketamine, as its corresponding hydrochloride salt, a compound of formula (I)

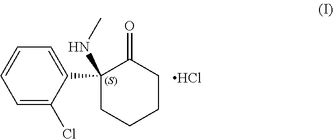

also known as (S)-2-(2-chlorophenyl)-2-(methylamino)cyclohexanone hydrochloride.

In an embodiment, the present invention is directed to methods for the treatment of treatment-refractory or treatment-resistant depression, wherein the esketamine is administered at a dosage amount in the range of from about 0.01 mg to about 1000 mg, or any amount or range therein, preferably from about 0.01 mg to about 500 mg, or any amount or range therein, preferably from about 0.1 mg to about 250 mg, or any amount or range therein. In another embodiment, the present invention is directed to methods for the treatment of treatment-refractory or treatment-resistant depression, wherein the esketamine is administered at a dosage amount in the range of from about 0.01 mg to about 1000 mg, preferably selected from the group consisting of 0.01 mg, 0.025 mg, 0.05 mg, 0.1 mg, 0.5 mg, 1 mg, 5 mg, 10 mg, 25 mg, 50 mg, 100 mg, 150 mg, 200 mg, 250 mg and 500 mg.

As used herein, unless otherwise noted, the term “antidepressant” shall mean any pharmaceutical agent which can be used to treat depression. Suitable examples include, but are not limited to mono-amine oxidase inhibitors such as phenelzine, tranylcypromine, moclobemide, and the like; tricyclics such as imipramine, amitriptyline, desipramine, nortriptyline, doxepin, protriptyline, trimipramine, chlomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, citolapram, escitolapram, fluvoxamine, and the like; serotonin receptor antagonists such as nefazadone, and the like; serotonin noradrenergic reuptake inhibitors such as venlafaxine, milnacipran, desvenlafaxine, duloxetine and the like; noradrenergic and specific serotonergic agents such as mirtazapine, and the like; noradrenaline reuptake inhibitors such as reboxetine, edivoxetine and the like; atypical antidepressants such as bupropion, and the like; lithium, natural products such as Kava-Kava, St. John's Wort, and the like; dietary supplements such as s-adenosylmethionine., and the like; and neuropeptides such as thyrotropin-releasing hormone and the like; compounds targeting neuropeptide receptors such as neurokinin receptor antagonists and the like; and hormones such as triiodothyronine, and the like. Preferably, the antidepressant is selected from the group consisting of fluoxetine, imipramine, bupropion, venlafaxine and sertaline.

Therapeutically effective dosage levels and dosage regimens for antidepressants (for example, mono-amine oxidase inhibitors, tricyclics, serotonin reuptake inhibitors, serotonin noradrenergic reuptake inhibitors, noradrenergic and specific serotonergic agents, noradrenaline reuptake inhibitor, natural products, dietary supplements, neuropeptides, compounds targeting neuropeptide receptors, hormones and other pharmaceutical agents disclosed herein), may be readily determined by one of ordinary skill in the art. For example, therapeutic dosage amounts and regimens for pharmaceutical agents approved for sale are publicly available, for example as listed on packaging labels, in standard dosage guidelines, in standard dosage references such as the Physician's Desk Reference (Medical Economics Company or online at http:///www.pdrel.com) or other sources.

As used herein the term “antipsychotic” includes, but is not limited to:

(a) typical or traditional antipsychotics, such as phenothiazines (e.g., chlorpromazine, thioridazine, fluphenazine, perphenazine, trifluoperazine, levomepromazin), thioxanthenes (e.g., thiothixene, flupentixol), butyrophenones (e.g., haloperidol), dibenzoxazepines (e.g., loxapine), dihydroindolones (e.g., molindone), substituted benzamides (e.g., sulpride, amisulpride), and the like; and

(b) atypical antipsychotics, such as paliperidone, clozapine, risperidone, olanzapine, quetiapine, zotepine, ziprasidone, iloperidone, perospirone, blonanserin, sertindole, ORG-5222 (Organon), and the like; and others such as sonepiprazole, aripiprazole, nemonapride, SR-31742 (Sanofi), CX-516 (Cortex), SC-111 (Scotia), NE-100 (Taisho), and the like.

In an embodiment, the “atypical antipsychotic” is selected from the group consisting of aripiprazole, quetiapine, olanzapine, risperidone and paliperidone. In another embodiment, the atypical antipsychotic is selected from the group consisting of aripiprazole, quetiapine, olanzapine and risperidone; preferably, the atypical antipsychotic is selected from the group consisting of aripiprazole, quetiapine and olanzapine.

As used herein, the term “treatment-refractory or treatment-resistant depression” and the abbreviation “TRD” shall be defined as major depressive disorder that does not respond to adequate courses of at least two antidepressants, preferably two or more antidepressants, more preferably two to three, antidepressants.

One skilled in the art will recognize that the failure to respond to an adequate course of a given antidepressant may be determined retrospectively or prospectively. In an embodiment, at least one of the failures to respond to an adequate course of antidepressant is determined prospectively. In another embodiment, at least two of the failures to respond to an adequate course of antidepressant are determined prospectively. In another embodiment, at least one of the failures to respond to an adequate course of antidepressant is determined retrospectively. In another embodiment, at least two of the failures to respond to an adequate course of antidepressant are determined retrospectively.

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, or eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

Wherein the present invention is directed to therapy with a combination of agents, “therapeutically effective amount” shall mean that amount of the combination of agents taken together so that the combined effect elicits the desired biological or medicinal response. For example, the therapeutically effective amount of combination therapy comprising esketamine and a serotonin reuptake inhibitor would be the amount of esketamine and the amount of the serotonin reuptake inhibitor that when taken together or sequentially have a combined effect that is therapeutically effective, and may have a combined effect that is synergistic. Further, it will be recognized by one skilled in the art that in the case of combination therapy with a therapeutically effect amount, the amount of each component of the combination individually may or may not be therapeutically effective.

Wherein the present invention is directed to the administration of a combination, the compounds may be co-administered simultaneously, sequentially, separately or in a single pharmaceutical composition. Where the compounds are administered separately, the number of dosages of each compound given per day, may not necessarily be the same, e.g. where one compound may have a greater duration of activity, and will therefore, be administered less frequently. Further, the compounds may be administered via the same or different routes of administration, and at the same or different times during the course of the therapy, concurrently in divided or single combination forms. The instant invention is therefore understood as embracing all regimens of simultaneous or alternating treatment and the term “administering” is to be interpreted accordingly.

As used herein, the terms “co-therapy”, “combination therapy”, “adjunctive treatment”, “adjunctive therapy” and “combined treatment” shall mean treatment of a patient in need thereof by administering esketamine in combination with one or more antidepressant(s), and further, optionally in combination with one or more atypical antipsychotics wherein the esketamine and the antidepressant(s) are administered by any suitable means, simultaneously, sequentially, separately or in a single pharmaceutical formulation. Where the esketamine and the antidepressant(s) are administered in separate dosage forms, the number of dosages administered per day for each compound may be the same or different. The esketamine and the antidepressant(s) may be administered via the same or different routes of administration. Examples of suitable methods of administration include, but are not limited to, oral, intravenous (iv), intranasal (in) intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The esketamine and the antidepressant(s) may be administered according to simultaneous or alternating regimens, at the same or different times during the course of the therapy, concurrently in divided or single forms.

Optimal dosages to be administered may be readily determined by those skilled in the art, and will vary with the particular compound or compounds used, the mode of administration, the strength of the preparation and the advancement of the disease condition. In addition, factors associated with the particular patient being treated, including patient's sex, age, weight, diet, time of administration and concomitant diseases/medications, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

As used herein, unless otherwise noted, the terms “subject” and “patient” refer to an animal, preferably a mammal, most preferably a human, who has been the object of treatment, observation or experiment. Preferably, the subject or patient has experienced and/or exhibited at least one symptom of the disease or disorder to be treated and/or prevented.

As used herein, the term “composition” is intended to encompass a product comprising the specified ingredients in the specified amounts, as well as any product which results, directly or indirectly, from combinations of the specified ingredients in the specified amounts.

As used herein, the term “continuation therapy” has been applied to the continuation of antidepressants following acute treatment which ought to be routine for some months, with the purpose of preventing relapse. As used herein, the term “maintenance treatment” is a treatment given after a patient has responded to a previous treatment and is a longer treatment aimed at preventing recurrence in those patients at high risk.

The present invention further comprises pharmaceutical compositions for the treatment of treatment-refractory or treatment-resistant depression (TRD) containing esketamine, optionally in combination with one or more antidepressants, with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, esketamine, and optionally, at least one antidepressant, as the active ingredient(s) are intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful, and the like, of from about 0.01 mg to about 1000 mg or any amount or range therein, and may be given at a dosage of from about 0.01 mg/kg to about 1.5 mg/kg, or any amount or range therein, preferably from about 0.01 mg/kg/day to about 0.75 mg/kg, or any amount or range therein, preferably from about 0.05 mg/kg to about 0.5 mg/kg, or any amount or range therein, preferably from about 0.1 mg/kg to about 0.5 mg/kg, or any amount or range therein, of each active ingredient. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.01 mg to about 1,000 mg, or any amount or range therein, of each active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating treatment-refractory or treatment-resistant depression described in the present invention may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein; preferably from about 0.05 mg to about 500 mg of the compound, or any amount or range therein, of each active ingredient, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, esketamine, optionally in combination with at least one antidepressant, as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

The following Examples are set forth to aid in the understanding of the invention, and are not intended and should not be construed to limit in any way the invention set forth in the claims which follow thereafter.

### Example 1

### Efficacy of Esketamine Clinical Trial—Prophetic Example

The ability of the esketamine to treat treatment-refractory or treatment-resistant depression (TRD) may be evaluated via a suitably designed clinical study, as briefly summarized below. A copy of the complete clinical trial protocol is attached herewith.

### Clinical Study Design

The study is designed as a double-blind, double-randomization, placebo-controlled, multiple dose titration study in 30 adult subjects with treatment-resistant depression (TRD). The study consists of 3 phases: a screening phase of up to 2 weeks, a 7-day double-blind treatment phase (Day 1 to Day 7), and a 4-week post-treatment (follow up) phase.

Screening Phase:

All subjects undergo a screening period of approximately 2 weeks, which provides adequate time to assess their eligibility per inclusion/exclusion criteria for the study.

Treatment Phase:

On Day 1 of the treatment phase, a target of 30 adult subjects with TRD are enrolled and randomized to one of three treatment groups (Group 1: esketamine @0.40 mg/kg, Group 2: esketamine @0.20 mg/kg, or Group 3: placebo, i.v. infusion). If esketamine @0.40 mg/kg dose is not well tolerated on Day 1 and/or Day 4, the dose may be reduced to 0.3 mg/kg.

Subjects who have a reduction in MADRS total score of >50% versus baseline on Day 2, 3, or 4 (prior to dosing) are considered responders. Subjects who are responders after the dose on Day 1 receive the same treatment again on Day 4. For subjects who are not responders after the dose on Day 1, treatment on Day 4 is selected as follows: (a) If the subject was treated with Placebo on Day 1: the subject is then re-randomization to esketamine 0.40 mg/kg or esketamine 0.20 mg/kg i.v. infusion on Day 4; (b) If the subject was treated with Esketamine 0.20 mg/kg on Day 1: the subject is then assigned to treatment with esketamine 0.40 mg/kg i.v. infusion on Day 4; (c) If the subject was treated with Esketamine 0.40 mg/kg on Day 1: the subject is then assigned to treatment with esketamine 0.40 mg/kg i.v. infusion again on Day 4.

Follow-Up Phase:

One week (7 days) after the end of the double-blind treatment phase (Day 14), subjects return to the unit for a follow-up visit. Additionally, telephone visits are conducted 3 (i.e., Day 10), 10 (i.e., Day 17), 14 (i.e., Day 21), 21 (i.e., Day 28), and 28 (i.e., Day 35) days after the end of the double-blind treatment phase. The interval between the first and last dose of study medication is 3 days. The total study duration for each subject is a maximum of 7 weeks. The end of study is defined as the date of the last study assessment of the last subject in the trial.

### Clinical Assessment of Efficacy

The primary efficacy evaluation is the Montgomery-Asberg Depression Rating Scale (MADRS) total score including modified versions for 24-hours and 2-hours recall. Secondary evaluations include evaluation of (a) MDD symptoms using the Quick Inventory of Depressive Symptomatology-Self Report-16-item (7-days recall) with modified 14-item (24-hours recall) and 10-item (2-hours recall) versions; (b) the severity of illness based on the Clinical Global Impression—Severity (CGI-S) and the global change in major depressive disorder (MDD) based on the Clinical Global Impression—Improvement (CGI-I); (c) the severity of illness based on subject's impression using the PGI-S; and (d) patient perspective of global change in MDD since start of study treatment, as measured by PG I-C.

Additional clinical evaluations include PK venous blood samples for measurement of esketamine and noresketamine plasma concentrations, with a first PK sample on Day 1 (to evaluate the single-dose PK of esketamine) and an additional PK sample collected at 40 minutes after the start of the intravenous infusion on Day 4 (to evaluate the maximum esketamine concentrations, which are expected to be achieved at the end of the infusion). Physical examination, body weight, vital signs, digital pulse oximetry, 12-lead ECG, continuous ECG monitoring, clinical laboratory tests (chemistry, hematology, urinalysis), and evaluation of adverse events are performed throughout the study to monitor subject safety. An optional pharmacogenomic blood sample (10 mL) is collected to allow for pharmacogenomic research. The collection of adverse events and recording of concomitant therapies is started after the informed consent has been signed and continues until the final follow up assessment. Other safety evaluations include the C-SSRS (to assess risk of suicide), BPRS (to assess severity of emergent psychotic symptoms), MGH-CPFQ (to assess cognitive and executive dysfunction) and the CADSS (to assess severity of emergent dissociative symptoms).

### Results/Analysis

The primary endpoint is the change in the MADRS total score from Day 1 to Day 2 (24 hours after the first infusion). The primary comparison is between each esketamine treatment group and the placebo treatment group.

A mixed-effects model using repeated measures (MMRM) is performed on the change from baseline in MADRS total score up to the 2^{nd} infusion on Day 4. The model includes baseline score as covariate, and day, treatment, center and day-by-treatment interaction as fixed effects, and a random subject effect. Appropriate contrasts are used to determine the estimated differences between each esketamine dose and placebo. The contrast on Day 2 changes is of primary interests, and tested one-sidedly at the alpha level of 0.10.

Subjects who have a reduction in MADRS total score of >50% versus baseline on Day 2, 3, or 4 (prior to dosing) are considered responders. The response rate in each esketamine group are compared with placebo using the exact Mantel-Haenszel test stratified by center as a secondary analysis. Similar analyses are performed on secondary efficacy endpoints. The results of the 2^{nd} infusion is combined with the 1^{st} infusion, and explored with a similar mixed-model analysis.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed:

1. A method for the treatment of treatment-refractory or treatment-resistant depression comprising administering to a patient in need thereof, a therapeutically effective amount of esketamine.

2. A method as in claim 1, wherein the esketamine is administered in an amount in the range of from about 0.01 mg/kg to about 1.5 mg/kg.

3. A method as in claim 2, wherein the esketamine is administered in an amount in the range of from about 0.01 mg/kg to about 0.75 mg/kg.

4. A method as in claim 3, wherein the esketamine is administered in an amount in the range of from about 0.05 mg/kg to about 0.5 mg/kg.

5. A method as in claim 4, wherein the esketamine is administer intravenously in an amount of about 0.2 mg/kg or in an amount of about 0.4 mg/kg.

6. A method as in claim 1, wherein the esketamine is administered intravenously.

7. A method as in claim 1, wherein the esketamine is administered intranasally.

8. A method for the treatment of treatment-refractory or treatment-resistant depression comprising administering to a patient in need thereof, a therapeutically effective amount of combination therapy comprising esketamine and at least one antidepressant.

9. A method as in claim 8, wherein the combination therapy comprises esketamine and one to two antidepressants.

10. A method as in claim 8, wherein each antidepressant is independently selected from the group consisting of imipramine, amitriptyline, desipramine, nortriptyline, doxepin, protriptyline, trimipramine, maprotiline, amoxapine, trazodone, bupropion, chlomipramine, fluoxetine, duloxetine, escitalopram, citalopram, sertraline, paroxetine, fluvoxamine, nefazadone, venlafaxine, milnacipran, reboxetine, lithium, mirtazapine, phenelzine, tranylcypromine, moclobemide, Kava-Kava, St. John's Wart, s-adenosylmethionine, thyrotropin releasing hormone, neurokinin receptor antagonists and triiodothyronine.

11. A method as in claim 8, wherein each antidepressant is independently selected from the group consisting of mono-amine oxidase inhibitors, tricyclics, serotonin reuptake inhibitors, serotonin noradrenergic reuptake inhibitors; noradrenergic and specific serotonergic agents and atypical antidepressants.

12. A method as in claim 8, wherein each antidepressant is independently selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, sertraline, paroxetine, citalopram, fluvoxamine, venlafaxine, milnacipran, mirtazapine and bupropion.

13. A method as in claim 8, wherein the combination therapy comprises esketamine and one to two antidepressants independently selected from the group consisting of fluoxetine, imipramine, bupropion, venlafaxine and sertaline.

14. A method as in claim 8, wherein the combination therapy comprising esketamine and at least one antidepressant further comprises an atypical antidepressant.

15. A method as in claim 14, wherein the atypical antidepressant is selected from the group consisting of aripiprazole, quetiapine, olanzapine, risperidone and paliperidone.

16. A method as in claim 14, wherein the atypical antidepressant is selected from the group consisting of aripiprazole, quetiapine and olanzapine.

17. A pharmaceutical composition for the treatment of treatment-refractory or treatment-resistant depression comprising esketamine, optionally at least one antidepressant, and a at least one pharmaceutically acceptable carrier.

18. The use of esketamine in the preparation of a medicament for the treatment of treatment-refractory or treatment-resistant depression, in a patient in need thereof.

19. Esketamine for use in a method for the treatment of treatment-refractory or treatment-resistant depression, in a patient in need thereof.

20. A composition comprising esketamine for the treatment of treatment-refractory or treatment-resistant depression.
